(19) United States
(12) Patent Application Publication
(10) Pub. No.: US 2019/0055350 A1
(43) Pub. Date: Feb. 21, 2019

(54) AMINE-FUNCTIONAL POLYMERS AND METHODS FOR PRODUCING SUCH POLYMERS

(71) Applicants: Henkel AG & Co. KGaA, Duesseldorf (DE); Henkel IP & Holding GmbH, Duesseldorf (DE)

(72) Inventors: Jose Garcia-Miralles, Duesseldorf (DE); Hans-Georg Kinzelmann, Pulheim (DE); Yongxia Wang, Bridgewater, NJ (US); Olaf Hartmann, Duesseldorf (DE)

(21) Appl. No.: 16/166,843

(22) Filed: Oct. 22, 2018

Related U.S. Application Data
(63) Continuation of application No. PCT/EP2017/060165, filed on Apr. 28, 2017.

(30) Foreign Application Priority Data
Apr. 29, 2016 (EP) 16167691.1

(51) Int. Cl. C08G 63/91; C07C 229/30; C07C 229/08

(52) U.S. Cl. CPC C08G 63/91; C07C 229/30; C07C 2601/14; C08G 2190/00; C07C 229/08

(57) ABSTRACT

A process for producing a β-amino ester functionalized oligomer or polymer, said process comprising the steps of: providing a polyol represented by the formula A-(OH)_{q} wherein q≥2 and A denotes an oligomeric or polymeric backbone, and converting said polyol into its corresponding acetoacetate functionalized compound by transacetoacetylation with an acetoacetate reagent; and, subjecting said acetoacetate functionalized compound to either indirect amination or direct reductive amination. Said indirect amination may be characterized by comprising the steps of: converting the acetoacetate functionalized compound into its corresponding enamine by reaction with at least one amine bearing at least a primary or secondary amine group; and, reducing the enamine product of the previous step to form the corresponding β-amino ester functionalized compound.

### FIELD OF THE INVENTION

The present invention relates to a method of producing amine-functional oligomers or polymers. More particularly, the present invention pertains to a process for producing storage-stable, β-amino ester functional oligomers or polymers, said process comprising the formation of an intermediate acetoacetate functionalized compound from an oligomeric or polymeric polyol provided as the starting material of said process.

### BACKGROUND TO THE INVENTION

The present invention is concerned with the provision of reactive curing agents or hardeners which are intended to promote and/or control the curing reaction of polymers contained with coating, adhesives, sealant and elastomer (CASE) compositions. It will be recognized that amine-functional compounds have found significant utility as reactive hardeners or curing agents in this context, primarily by virtue of the amine functionality being reactive with inter alia: epoxides; isocyanates; amide/formaldehyde and other aldehyde condensates (aminoplasts); Michael acceptors; aziridines; acetylacetates; anhydrides; lactones and other active esters; ketenes and ketene dimers; aldehydes and ketones; coordinating transition metals; alkylating agents or their polymeric equivalents; and, acid halides. This list of amine reactive compounds and functionalities is not exhaustive.

Broadly, amine hardeners fall within four main groups: aliphatic amines; polyamides and amidoamines; cycloaliphatic amines; and, aromatic amines. There are, of course, relative performance differences amongst different amine hardeners which can either detract from or enhance the performance of the coating, adhesive, sealant or elastomer compositions for which these hardeners are utilized. Relative performance differences are manifested in terms of the color stability, viscosity, low temperature cure, water sensitivity, film flexibility, solvent resistance and acid resistance which the amine hardeners possess or impart.

The provision of the reactive amine functionality group on a polymeric or oligomeric backbone is known and the backbone polymer can moderate the performance of the reactive curing agent or hardener. For example, as compared to simple aliphatic amines, polyetheramines generally provide good color stability, good flexibility and reduced carbonation tendencies. However, because known polyetheramines also tend to react more slowly than simple aliphatic amines and also tend to be prone to attack by oxygenated solvents, there clearly remains a need in the art to further develop this polymer chemistry. Moreover, it would be advantageous to provide amine-functional polymers of other chemistries, such as polyesters for instance, which can allow for the development of improved or optimized reactive curing agents for specific coating, adhesive, sealant or elastomer applications.

It is known that the synthesis of amine functional polymers is, however, difficult for at least two reasons. The simplest amine functional monomer, vinylamine, is thermodynamically and kinetically unstable relative to the isomeric Schiff base and the condensation product of the base, ethylidine imine. Secondly, more stable allyl- and diallyl/l amine monomers are expensive and typically show severe chain transfer during free radical polymerization, especially when involving allyl protons on carbon atoms alpha to the nitrogen atom in the amine. The allylamines are known to produce mainly low molecular weight polymers and copolymers even when using large amounts of free radical initiators.

Given this, the inventors have focused on post-polymerization modification of hydroxyl functional oligomers and polymers in order to engineer synthetic polymers bearing amine functional groups, as a practical alternative to polymerization and copolymerization strategies. The difficulty of such post-polymerization modification has also been identified in the prior art, however.

Li et al. Synthesis of Linear Polyether Polyol Derivatives as New Materials for Bioconjugation Bioconjugate Chem. 2009, 20, 780-789, describes a method of amino functionalization by the post-polymerization modification of the hydroxyl groups of linPG-co-PEO. This reported method showed a limited overall conversion for amino functions of only 34%.

EP 2162683 A2 (Evonik Degussa GMBH) describes a process for preparing an amino group containing polyester which comprises reacting a polyester with one or more polyamines having at least one primary and at least one secondary amino group. It is considered that the transamidation reaction described in this document is very unselective and “chops” the polyester backbone; this leads to a complex mixture of products having polymeric or oligomeric backbones of different lengths and which comprise both amino and hydroxyl groups.

U.S. Pat. No. 5,525,683 (Adkins et al.) describes a process for the production of an ether-linked amine-terminated polyester comprising reacting: 1) a polyester polyol in which substantially all of the hydroxyl groups have been converted to a leaving group; with 2) an aminoalcohol and/or aminothiol; and, 3) a material which is capable of deprotonating aminoalcohol and/or aminothiol 2). In this document's singular exemplified embodiment, step 1) of the process comprises converting the hydroxyl groups of a polycaprolactone polyester polyol to methanesulfonate leaving groups by reaction of said polyester polyol with methane sulfonyl chloride in the presence of triethylamine and methylene chloride.

EP 0 429 169 A1 (Imperial Chemical Industries PLC) describes a process for preparing an isocyanate-reactive polymer containing a plurality of enamine ester groups, which can be used in adhesives, coatings or elastomer compositions. A reduction of said enamine ester compound to a corresponding l-amino ester compound is not disclosed.

EP 0 477 697 A2 (Mobay Corporation) describes a process for the production of an enamine ester compound for use in a resin injection molding process (RIM). A reduction of the enamine ester compound to a corresponding L-amino ester compound is not disclosed.

To this point in the art, post-polymerization modifications to produce amine functional polymers and oligomers are often time-consuming, can involve sensitive reagents and can suffer from a limited overall conversion. Furthermore, the post-polymerization modifications may lead to amine functional polymers or oligomers with low storage stability, such as e.g. enamine ester functional polymers or oligomers.

### SUMMARY OF THE INVENTION

At its broadest, the present invention is directed to a process for producing a β-amino ester functionalized oligomer or polymer, said process comprising the formation of an intermediate acetoacetate functionalized compound from an oligomeric or polymeric polyol provided as the starting material of said process. That intermediate acetoacetate functionalized compound is then subjected to either indirect reductive amination (herein also denoted as “indirect amination”) or direct reductive amination.

In accordance with a first aspect of the invention, there is provided a process for producing a β-amino ester functionalized oligomer or polymer, said process comprising the steps of:

a) providing a polyol represented by the formula A-(OH)q wherein q≥2 and A denotes an oligomeric or polymeric backbone, and converting said polyol into its corresponding acetoacetate functionalized compound by transacetoacetylation with an acetoacetate reagent;

b) converting said acetoacetate functionalized compound into its corresponding enamine by reaction with at least one amine bearing at least a primary or secondary amine group; and,

c) reducing the enamine product of step b) to form the corresponding β-amino ester functionalized compound.

In accordance with a second aspect of the invention, there is provided a process for producing a β-amino ester functionalized oligomer or polymer, said process comprising the steps of:

a) providing a polyol represented by the formula A-(OH)q wherein q≥2 and A denotes an oligomeric or polymeric backbone, and converting said polyol into its corresponding acetoacetate functionalized compound by transacetoacetylation with an acetoacetate reagent; and,

d) converting said acetoacetate functionalized compound into its corresponding β-amino ester by a reductive amination with at least one amine bearing at least a primary or secondary amine group.

Said reductive amination step d) is preferably performed in the presence of a hydride as a hydrogen source. More particularly, the reductive amination is performed using an aluminium hydride or borohydride compound and, most preferably, said reductive amination is performed in the presence of: a borohydride comprising an anion having the formula [(X)nBH4-n]- wherein: n=0, 1, 2 or 3; and, X is a cyano, acetoxy, trifluoroacetoxy, C1-C6 alkoxy or C1-C6 alkyl group; or, an aluminium hydride comprising an anion having the formula [(X)nAlH4-n]- wherein: n=0, 1, 2 or 3; and X is a C1-C6 alkoxy or C1-C6 alkyl group.

The acetoacetate reagent employed in both said aspects may be represented by Formula 1 hereinbelow:

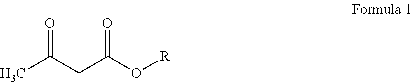

wherein R is a C1-C12 alkyl group, preferably a C1-C6 alkyl group.

As regards the polyol starting material, “A” thereof preferably denotes an oligomeric or polymeric backbone with hetero atoms in the backbone or in pendent side chains. A particular preference is mentioned for polyols selected from the group consisting of: polyoxyalkylene polyols; polyester polyols; polycarbonate polyols; and, mixtures thereof. The hydroxyl functionality, q, of said polyol is typically from 2 to 6 and preferably from 2 to 4. The polyol will typically have a number average molecular weight (Mn) of from 300 to 10000 g/mol, preferably from 400 to 9000 g/mol, more preferably from 500 to 8000 g/mol, even more preferably from 1000 to 6000 g/mol. These preferred properties of the polyol are not mutually exclusive; the polyols may be characterized by combinations of said properties.

The at least one amine employed in step b) or step d) of the above defined processes is commonly represented by Formula 3 herein below:

R²R³NH Formula 3

wherein: R² is hydrogen or a C1-C6 alkyl group;
R³ is hydrogen or a C1-C18 aliphatic hydrocarbyl group which is optionally interrupted by one or more —N(R⁴)— groups of which R⁴ is a hydrogen atom; and,
R² and R³ may form a ring together with the N-atom to which they are bound.

In a first embodiment, an amine reactant is provided in which R² is hydrogen and R³ is a C1 to C12 alkyl group, preferably a C1 to C6 alkyl group.

In an independent embodiment, an amine reactant is provided wherein R² is hydrogen and, R³ is a C1 to C18 hydrocarbyl group, preferably a C1 to C12 hydrocarbyl group which is interrupted by one or more —N(R⁴)— groups of which R⁴ is a hydrogen atom.

The above defined processes have been found to be highly selective. By virtue of which, these processes have enabled the formation of β-amino ester functional oligomer or polymers which are characterized by: a primary amine level of less than 5 mg KOH/g, preferably less than 1 mg KOH/g; and, a secondary amine level of from 5 to 599 mg KOH/g, preferably from 5 to 300 mg KOH/g. Moreover, these processes retain the integrity of the polymeric backbone which is not therefore spliced or reduced in molecular weight. The polydispersity of the β-amino ester functional compounds corresponds substantially to that of the starting polyol.

In accordance with a further aspect of the invention, there is provided a β-amino ester functional oligomer or polymer obtained by the aforementioned process.

The use of the β-amino ester functional oligomers or polymers as hardeners or reactive curing agents for coating, adhesive, sealant or elastomer compositions based on compounds bearing amine-reactive functionalities, in particular compounds bearing amine-reactive functionalities selected from epoxy groups, isocyanate groups and cyclic carbonate groups, is an additional important aspect of the present invention. Advantageously, as regards this utility, the β-amino ester functional oligomers or polymers of the present invention show storage stability.

### Definitions

Unless otherwise stated, the term “molecular weight” as used herein for oligomeric, polymeric and co-polymeric species refers to number average molecular weight (Mn) as determined by gel permeation chromatography (GPC) against a polystyrene standard.

The term “polyol” as used herein shall include diols and higher functionality hydroxyl compounds.

The hydroxyl (OH) number of a polyol is the quantity of potassium hydroxide in milligrams that is equivalent to the hydroxyl groups in 1 g of substance. The hydroxyl numbers given here are determined by acetylating hydroxyl groups in polyols and polyol systems with acetic anhydride and then titrating the excess acetic anhydride with alcoholic potassium hydroxide solution in accordance with DGF C-V 17a (53).

The amine values given herein are determined by titration with hydrochloric acid in accordance with ASTM D 2074-92 and thereafter calculated back to mg KOH.

As used herein, the term “aliphatic hydrocarbyl group” refers to a residue that contains only carbon and hydrogen atoms. As such, a C1 to C18 aliphatic hydrocarbyl residue contains from 1 to 18 carbons atoms. The residue may be straight chain, cyclic, bicyclic, branched, saturated or unsaturated. It may also contain combinations of straight chain, cyclic, bicyclic, branched, saturated or unsaturated moieties. When so stated, the hydrocarbyl residue may contain heteroatoms within the backbone thereof.

Unless otherwise indicated, the term “alkyl”, as used herein, includes straight chain moieties, and where the number of carbon atoms suffices, branched moieties. As such, the term “C1-C12 alkyl” includes both saturated straight chain and branched alkyl groups having from 1 to 12 carbon atoms. Analogously the term “C1-C6 alkyl” includes saturated straight chain and branched alkyl groups having from 1 to 6 carbon atoms. Examples of C1-C6 alkyl groups include methyl, ethyl, propyl, isopropyl, butyl, isobutyl, tert-butyl, pentyl and hexyl groups.

The term “C3-C6 cycloalkyl” as used herein means a saturated cyclic hydrocarbon having 3-6 carbon atoms, i.e. cyclopropyl, cyclobutyl, cyclopentyl or cyclohexyl.

The term “alkoxy”, as used herein, means “—O-alkyl” or “alkyl-O—”, wherein “alkyl” is defined as above.

As used herein, the term “interrupted by one or more” of a stated heteroatom means that the or each heteroatom may be positioned at any position along the hydrocarbyl chain including at either end of the chain.

As applied herein as a characterization of the β-amino ester functional polymer product, the term “a storage stable” means a product which has a level of free amines—determined by titration—after storage for 28 days at 40° C. which differs by no more than 20% from the initial level of amines determined by titration at day 0. In many embodiments, the β-amino ester functional polymers of the present invention also do not show any discoloration upon storage for 28 days at 40° C.

The term “β-amino ester” and “beta-amino ester” are used interchangeably.

### DETAILED DESCRIPTION OF THE INVENTION

Both aspects of the present invention as defined above proceed with a common preliminary step.

### Step a) Acetoacetate Functionalization of the Polyol

Step a) of the above defined process provides acetoacetate functionalized oligomers or polymers via a reaction which proceeds in accordance with the following equation (Reaction 1):

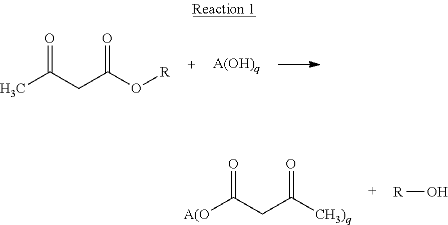

Reaction 1 above may be described as the transesterification—or more specifically the transacetoacetylation—of the polyols with an acetoacetate compound as defined in Formula 1 below:

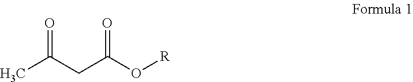

wherein R is a C1-C12 alkyl group. More typically, the constituent alkyl group R has from 1 to 8 and preferably from 1 to 6 carbon atoms. Exemplary alkyl acetoacetates include: t-butyl acetoacetate; isobutyl acetoacetate; n-butyl acetoacetate; isopropyl acetoacetate; n-propyl acetoacetate; ethyl acetoacetate; and, methyl acetoacetate t-butyl acetoacetate is preferred herein.

The polyol employed in Reaction 1 above is denoted by Formula 2 herein below:

A-(OH)_{q} Formula 2

wherein q≥2 and A denotes an oligomeric or polymeric backbone which preferably includes hetero atoms in the backbone or in pendent side chains. In one embodiment, the reactant polyol is characterized by: a number average molecular weight (Mn) of from 300 to 10000 g/mol, preferably from 400 to 9000 g/mol, more preferably from 500 to 8000 g/mol, even more preferably from 1000 to 6000 g/mol; and, a hydroxyl functionality, q, of from 2 to 6, preferably from 2 to 4. In a further independent or preferably complimentary embodiment, the reactant polyol is selected from the group consisting of: polyoxyalkylene polyols, also called polyether polyols; polyester polyols; polycarbonate polyols; polycaprolactone; polyacrylate polyols; polytetrahydrofuran (or polytetramethylene glycol, PTMEG) polyol; and, mixtures thereof. For example, the reactant polyol may be selected from the group consisting of: polyoxyalkylene polyols; polyester polyols; polycarbonate polyols; and, mixtures thereof. The use of one or more polyester polyols as the starting material is of particular interest.

As is known in the art, polyester polyols can be prepared from condensation reactions of polybasic carboxylic acids or anhydrides and a stoichiometric excess of polyhydric alcohols, or from a mixture of polybasic carboxylic acids, monobasic carboxylic acids and polyhydric alcohols. Suitable polybasic carboxylic acids and anhydrides for use in preparing the polyester polyols include those having from 2 to 18 carbon atoms and in particular those having from 2 to 10 carbon atoms. Non-limiting examples of such polybasic carboxylic acids and anhydrides include: adipic acid; glutaric acid; succinic acid; malonic acid; pimelic acid; sebacic acid; suberic acid; azelaic acid; 1,4-cyclohexane dicarboxylic acid; phthalic acid; phthalic anhydride; isophthalic acid; terephthalic acid; tetrahydrophthalic acid; hexahydrophthalic acid; and, combinations thereof. Monobasic carboxylic acids which can be used include those having from 1 to 18 carbon atoms or, preferably from 1 to 10 carbon atoms, of which the following examples might be mentioned: formic acid; acetic acid; propionic acid; butyric acid; valeric acid; caproic acid; caprylic acid; capric acid; lauric acid; myristic acid; palmitic acid; stearic acid; and, combinations thereof. Suitable polyhydric alcohols have from 2 to 18 carbon atoms and desirably from 2 to 10 carbon atoms. Exemplary polyhydric alcohols include, but are not limited to: ethylene glycol; propylene glycol; hexane-1,6-diol; trimethylol propane; glycerol; neopentyl glycol; pentaerythritol; butylene glycol; 2-methyl-1,3-propane diol; hexylene glycol; and, combinations thereof.

Polyether polyols may be produced by processes known in the art, such as the reaction of alkene oxides with polyhydric starter molecule in the presence of an appropriate catalyst, such as an alkali metal hydroxide, alkali metal alkoxide or antimony pentachloride. Examples of the alkene oxides include: tetrahydrofuran; ethylene oxide; 1,2-propylene oxide; 1,2- and 2,3-butylene oxide; and, styrene oxide. And examples of suitable starter molecules include but are not limited to: water; ethylene glycol; 1,2- and 1,3-propanediols; 1,4-butanediol; diethylene glycol; and, trimethylol-propane. Preferred polyether polyols for use herein are: poly(propylene oxide) polyol; poly(ethylene oxide) polyol; polytetramethylene ether glycol PTMEG; and, mixtures thereof.

Polycarbonate polyols for use herein can be selected from, but are not limited to polycarbonate diols. Such polycarbonate diols may be produced by the reaction of a diol with dialkyl or diaryl carbonates or phosgene. The reactant diols may be selected from, but are not limited to: 1,2-propanediol; 1,3-propanediol; 1,4-butanediol; 1,5-pentanediol; 1,6-hexanediol; diethylene glycol; triethylene glycol; and, mixtures thereof. An exemplary diaryl carbonate is diphenyl carbonate.

The transesterification (transacetoacetylation) Reaction 1 may be conducted by conventional methods as known in the art of polymer chemistry. Reference in this regard may be made to inter alia: Witzman et al. “Comparison of Methods for the Preparation of Acetoacetylated Coating Resins”, Journal of Coatings Technology, Vol. 62, No. 789, October 1990; and, Witzeman et al. “Transacetoacetylation with tert-butyl acetoacetate: Synthetic Applications”, J. Org. Chemistry 1991, 56, 1713-1718. Typically, the reaction between the oligomeric or polymeric polyol and the acetoacetate will involve mixing said polyol and acetoacetate in a suitable vessel, either with or without solvent, at an elevated temperature of, for example, from 50° to 200° C. or from 80° to 150° C.; preferably, the reaction is performed in the absence of solvent. The reaction is driven towards completion by distilling off the alcohol (R—OH) formed under reduced pressure. Moreover, the reaction is preferably conducted in the presence of a transesterification catalyst of which suitable examples include, but are not limited to, calcium acetate, zinc acetate, bismuth acetate, lead oxide and trichloroacetic acid.

The reaction should proceed to at least 99% conversion of the hydroxyl groups into acetoacetate functional groups. Whilst the reactants may be used in amounts such that one OH group is present for each acetoacetate group, it is also preferred to use a molar excess of the acetoacetate to ensure complete reaction.

The acetoacetate functionalized oligomers or polymers may be processed to yield amino-functional oligomers or polymers in either a two-step [b) and c)] process which proceeds via an intermediate enamine or a one-step (one-pot) [d)] process.

### Step b): Formation of Intermediate Enamines

The intermediate enamine resins of the present invention are prepared by reacting the acetoacetylated resin product of Reaction 1 with one or more aliphatic primary or secondary amine. In particular, the acetoacetylated resin product of Reaction 1 is reacted with one or more amines of Formula 3:

R²R³NH Formula 3

wherein: R² is hydrogen or a C1-C6 alkyl group;
R³ is hydrogen or a C1-C18 aliphatic hydrocarbyl group which is optionally interrupted by one or more —N(R⁴)— groups of which R⁴ is a hydrogen atom; and,
R² and R³ may form a ring together with the N-atom to which they are bound.
   For completeness, where R² and R³ form a ring, it will be recognized that such a ring may be heterocyclic in that it may include one or more nitrogen atoms.

In an embodiment, a reactant amine is a primary amine characterized in that R² is hydrogen and R³ is a C1 to C12 alkyl group, preferably a C1 to C6 alkyl group. Exemplary amines of this type include: n-butylamine; n-hexylamine; n-octylamine; n-decylamine; and, n-dodecylamine.

In a further embodiment, a reactant amine is characterized in that: R² is hydrogen; and, R³ is a C1 to C18 hydrocarbyl group, preferably a C1 to C12 hydrocarbyl group which is interrupted by one or more —N(R⁴)— groups of which R⁴ is a hydrogen atom. Exemplary di-primary amines of this embodiment include: tetramethylene diamine; pentamethylene diamine; hexamethylene diamine; octamethylene diamine; and, dodecamethylene diamine. Exemplary primary-secondary diamines of this embodiment include: N-methylethylenediamine; N-ethylethylenediamine; N-methyl-1,3-diaminopropane; 2-(isopropylamino)ethylamine; N-propylethylenediamine; N-propyl-1,3-propanediamine; N-cyclohexyl-1,3-propanediamine; 4-(aminomethyl)piperidine; 3-(aminomethyl)piperidine; 2-(aminomethyl)piperidine; and, 4-aminopiperidine.

Further exemplary amines suitable for use in the present invention include: piperidine; pyrollidine; and, N,N′-dimethyl-1,6-hexanediamine. At present, good results have in particular been obtained when the reactant amine comprises one or more of: N-methyl-1,3-diaminopropane; 4-(aminomethyl)piperidine; N-cyclohexyl-1,3-propanediamine; and, n-butylamine.

The reaction of step b) may be represented by the following generalized scheme (Reaction 2):

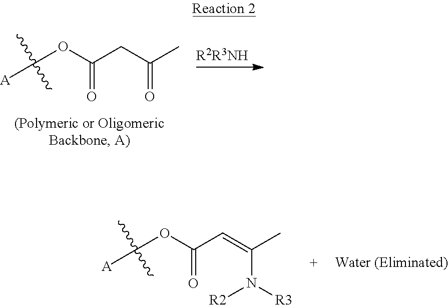

The amount of amine is generally selected so that one mole of amine is available for every acetoacetate equivalent. Small variances about a 1:1 equivalence ratio can however be tolerated and, as such the molar equivalence ration of acetoacetate to amine may be in the range from 0.8:1 to 1.2:1.

Generally the Reaction (2) is carried out under an inert atmosphere, for instance under nitrogen or argon gas, at a temperature of from 10° to 200° C. and preferably from 20° to 100° C. The performance of the process at room temperature is not therefore precluded.

Whilst it is not critical for solvents to be present in the course of the reaction, the presence of solvents that form azeotropes with the water also produced in the reaction can be beneficial. Exemplary solvents of this type include: dichloromethane; trichloromethane; chlorobenzene; dichlorobenzenes; toluene; xylene; ethylacetate; propylacetate; butylacetate; diethylether; and, dibutylether. When present, the amount of solvent is generally selected so as to be sufficient to dissolve the starting materials; this will typically to equate to the use of the solvent in an amount of from 20 to 500, and preferably from 50 to 200 parts by weight per 100 parts by weight of the acetoacetate functionalized polymer.

The progress of the reaction may be monitored by one or more of thin layer chromatography (TLC), amine titration and infrared (IR) spectroscopy. The reaction time will, of course, depend on the nature and the amounts of starting materials but commonly reaction times will fall between 1 and 10 or between 1 and 8 hours.

When the reaction is complete, the intermediate enamine product is isolated from the eliminated water and any unreacted amine. This may be effected by reduced pressure or vacuum distillation, whereby the distillate may be subjected to further processing to enable, for instance, the recycling of unreacted amine. Water may be removed either from the product of Reaction 2 or any distillate collected through the use of dehydrating agents, such as calcium oxide, sodium sulfate, and so-called molecular sieves.

### Step c): Reduction of the Enamine Intermediate

As described hereinbefore, the isolated intermediate enamine product is then reduced to the corresponding beta-amino ester in accordance with the following generalized reaction (Reaction 3):

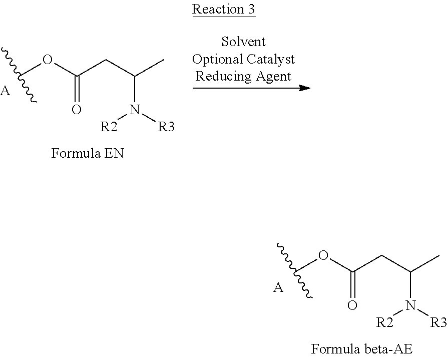

There is no particular intention to limit the reducing agents which may be used in this step of the process. In some embodiments, however, the reducing agent may be sodium borohydride, potassium borohydride, lithium borohydride, lithium triethylborohydride, zinc borohydride, aluminum borohydride, calcium borohydride, magnesium borohydride, sodium triacetoxyborohydride, tetramethylammonium triacetoxyborohydride, boranepyridine, 2-picoline borane, 9-borabicyclo(3.3.1)nonane, sodium or potassium triethylborohyride, sodium or potassium triphenylborohydride, lithium bis(triphenylphosphine)copper borohydride, lithium morphilinoborohydride, lithium pyrrolidinoborohydride, or sodium cyanoborohydride.

Whilst a person of ordinary skill in the art will be able to determine an appropriate amount of reducing agent for use in this step of the process, the molar ratio of the compound of Formula EN to the reducing agent will typically be in the range from 1:0.2 to 1:4 or from 1:0.5 to 1:3. Exemplary, but non-limiting, molar ratios which might be mentioned are from 1:0.5 to 1:2 and 1:0.8 to 1:2.

The reaction mixture further comprises one or more solvents, of which at least one said solvent is preferably miscible with water. It is therefore envisaged that the reaction may be performed in a solvent system consisting of two or more solvents that are miscible with water. Equally, the reaction may be performed in a solvent system consisting of at least one solvent that is immiscible with water and at least one solvent that is miscible with water. For completeness, the term “immiscible” as used herein means that in some proportion two phases are present.

Non-limiting examples of solvents miscible with water include, without limit, acetic acid, acetone, acetonitrile, dimethylformamide, dimethyl sulfoxide, dioxane, ethanol, methanol, n-propanol, isopropanol, and tetrahydrofuran. Non-limiting examples of solvents that are immiscible with water include benzene, n-butanol, butyl acetate, carbon tetrachloride, chloroform, cyclohexane, 1,2-dichloroethane, dichloromethane, ethyl acetate, di-ethyl ether, heptane, hexane, methyl-1-butyl ether, methyl ethyl ketone, pentane, di-isopropyl ether, toluene, trichloromethane, xylene, and combinations thereof.

The amount of solvent present during this step of the process may be determined based on normal practical considerations. In general, however, the volume to mass ratio of the solvent to the compound of Formula EN will be in the range from 1:1 to 100:1. In some embodiments, the volume to mass ratio of the solvent to the compound of Formula EN may be in range from 1:1 to 50:1.

Without specific intention to limit said conditions, the reduction step may be conducted at a temperature of from 00 to 120° C., preferably from 20° to 100° C. and for a sufficient period of time to allow the reaction to reach completion or to reach a point at which the amount of the enamine intermediate remaining in the reaction mixture—determinable by thin layer chromatography, for example—is less than 3 wt. % or less than 1 wt. %. Typically, the reaction duration will fall in the range of from 2 to 96 hours, for example from 3 to 48 hours. Thereafter, the reaction may be quenched by the addition of an appropriate weak base such as sodium hydrogen carbonate.

The identified beta amino esters of Formula β-AE above are isolated from the reaction mixture using techniques known to those of ordinary skill in the art. Mention in this regard may be made of extraction, evaporation, distillation and chromatography as suitable techniques. Upon isolation, it has been found that typical yields of the compound of Formula β-AE are at least 40% and often at least 60% or 80%.

### Step d) Direct Reductive Amination of the Acetoacetate Functionalized Polymer

In accordance with the second aspect of the invention as described hereinbefore, the beta-amino ester product may be produced in a one-step process from the acetoacetate functionalized polymer formed in Reaction 1 above. By performing a direct reductive amination of said polymer, one obviates the need to isolate an enamine intermediate.

The term “direct reductive amination”, as used herein, refers to a process whereby the acetoacetate functionalized compound—the product of Reaction 1—is combined with ammonia, an ammonia source, a primary amine, a secondary amine or a primary/secondary amine, such that the compounds condense to generate an intermediate imine or iminium ion that may be subjected to reduction by means of hydrogenation. Said hydrogenation may be mediated by a metal catalyst and requires a hydrogen source such as hydrogen gas or a precursor thereof including but, not limited to, formate derivatives, cyclohexadiene and other hydride sources.

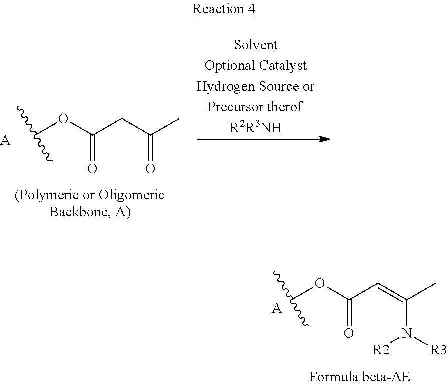

The reactant amines may be characterized by meeting Formula 3 hereinbelow:

R²R³NH Formula 3

wherein: R² is hydrogen or a C1-C6 alkyl group;
R³ is hydrogen or a C1-C18 aliphatic hydrocarbyl group which is optionally interrupted by one or more —N(R⁴)— groups of which R⁴ is a hydrogen atom; and,
R² and R³ may form a ring together with the N-atom to which they are bound.
   For completeness, where R² and R³ form a ring, it will be recognized that such a ring may be heterocyclic in that it may include one or more nitrogen atoms.

In an embodiment, a reactant amine is a primary amine characterized in that R² is hydrogen and R³ is a C1 to C12 alkyl group, preferably a C1 to C6 alkyl group. Exemplary amines of this type include: n-butylamine; n-hexylamine; n-octylamine; n-decylamine; and, n-dodecylamine.

In a further embodiment, a reactant amine is characterized in that: R² is hydrogen; and, R³ is a C1 to C18 hydrocarbyl group, preferably a C1 to C12 hydrocarbyl group which is interrupted by one or more —N(R⁴)— groups of which R⁴ is a hydrogen atom. Exemplary di-primary amines of this embodiment include: tetramethylene diamine; pentamethylene diamine; hexamethylene diamine; octamethylene diamine; and, dodecamethylene diamine. Exemplary primary-secondary diamines of this embodiment include: N-methylethylenediamine; N-ethylethylenediamine; N-methyl-1,3-diaminopropane; 2-(isopropylamino)ethylamine; N-propylethylenediamine; N-propyl-1,3-propanediamine; N-cyclohexyl-1,3-propanediamine; 4-(aminomethyl)piperidine; 3-(aminomethyl)piperidine; 2-(aminomethyl)piperidine; and, 4-aminopiperidine.

Further exemplary amines suitable for use in the present invention include: piperidine; pyrollidine; and, N,N′-dimethyl-1,6-hexanediamine. At present, good results have in particular been obtained when the reactant amine comprises one or more of: N-methyl-1,3-diaminopropane; 4-(aminomethyl)piperidine; N-cyclohexyl-1,3-propanediamine; and, n-butylamine.

The amount of amine is generally selected so that one mole of amine is available for every acetoacetate equivalent. Small variances about a 1:1 equivalence ratio can however be tolerated and, as such the molar equivalence ratio of acetoacetate to amine may be in the range from 0.8:1 to 1.2:1.

In an embodiment of Reaction 4, a hydride reagent is employed and it is therefore noted that suitable hydride reagents for use herein include: silanes; stannanes; and, preferably, boron or aluminum hydride sources. Particularly suitable borohydrides are those comprising an anion of the formula [(X)ₙBH₄₋ₙ]— wherein: n=0, 1, 2 or 3; and, X is a cyano, acetoxy, trifluoroacetoxy, C1-C6 alkoxy or C1-C6 alkyl group. The counter-ion present in such borohydride will, typically, be Li⁺, Na⁺, K⁺ or NH₄⁺. In this regard the attention of the reader is directed to Abdel-Magid et al. “Reductive Amination of Aldehydes and Ketones with Sodium Triacetoxyborohydride” Journal of Organic Chemistry, 1996, 61, 3849-3862. Particularly suitable aluminium hydrides are those comprising an anion of the formula [(X)ₙAlH₄₋ₙ]⁻ wherein: n=0, 1, 2 or 3; and X is a C1-C6 alkoxy or C1-C6 alkyl group. The counter-ion present in such aluminium hydride may be Na⁺, K⁺, NH₄⁺ or preferably Li⁺.

The amount of hydride is generally selected such that the molar equivalence ratio of hydride to amine is in the range from 1:1 to 2:1, preferably from 1.2:1 to 1.8:1 and more preferably from 1.3:1 to 1.6:1.

In a preferred embodiment of Reaction 4, hydrogen (H₂) is used in the presence of a hydrogenation catalyst. Suitable catalysts may be found, for instance, in: Houben-Weyl Methoden der Organischen Chemie, 4th Edition, Vol. 11/1, page 602; and, Handbook of Heterogeneous Catalysis, 2nd Edition, Vol. 7, 2008, Wiley VCH, page 3554. As non-limiting examples of reductive amination catalysts, there might be mentioned: Raney nickel; nickel; palladium; Lindlar catalyst; cobalt; copper chromite; platinum; platinum oxide; rhenium; tin(II) chloride; titanium(III) chloride; zinc; iron; and, mixtures thereof. Herein a particular preference is given to palladium, cobalt and ruthenium. More particularly, good results have been obtained when palladium is used as a hydrogenation catalyst.

As is known in the art, the aforementioned catalysts may be used as such or may be applied to an appropriate support, such as aluminum oxide, silicon dioxide, titanium dioxide, zirconium dioxide and activated carbon.

Where used, the amount of hydrogenating catalyst—as determined in the absence of any support—should be from 0.001 to 10 wt. %, preferably from 0.01 to 5 wt. % by weight, based on the total weight of reactant amine used.

The reaction mixture further comprises one or more solvents, of which at least one said solvent is preferably miscible with water. It is therefore envisaged that the reaction may be performed in a solvent system consisting of two or more solvents that are miscible with water. Equally, the reaction may be performed in a solvent system consisting of at least one solvent that is immiscible with water and at least one solvent that is miscible with water. For completeness, the term “immiscible” as used herein means that in some proportion two phases are present.

Non-limiting examples of solvents miscible with water include, without limit, acetic acid, acetone, acetonitrile, dimethylformamide, dimethyl sulfoxide, dioxane, ethanol, methanol, n-propanol, isopropanol, and tetrahydrofuran. Non-limiting examples of solvents that are immiscible with water include benzene, n-butanol, butyl acetate, carbon tetrachloride, chloroform, cyclohexane, 1,2-dichloroethane, dichloromethane, ethyl acetate, di-ethyl ether, heptane, hexane, methyl-1-butyl ether, methyl ethyl ketone, pentane, di-isopropyl ether, toluene, trichloromethane, xylene, and combinations thereof.

The amount of solvent present during this step of the process may be determined based on normal practical considerations. In general, however, the volume to mass ratio of the solvent to the compound of acetoacetate functionalized will be in the range from 1:1 to 100:1. In some embodiments, the volume to mass ratio of the solvent to the acetoacetate functionalized compound may be in range from 1:1 to 50:1.

Without specific intention to limit said conditions, the reductive amination may be conducted at a temperature of from 0° to 120° C., preferably from 20° to 100° C. and for a sufficient period of time to allow the reaction to reach completion or to reach a point at which Typically, the reaction duration will fall in the range of from 2 to 96 hours, for example from 3 to 48 hours. Thereafter, the reaction may be quenched by the addition of an appropriate weak base such as sodium hydrogen carbonate.

Upon completion of the reductive amination, it is possible to remove any solid, suspended hydrogenation catalyst by, for example, filtration, crossflow filtration or centrifugation. Such a separation step is not necessary where the catalyst was disposed in a fixed bed; in this circumstance the hydrogenation output is simply removed from the reaction vessel. The catalyst can be recycled with appropriate compensation for the loss of catalyst through attrition and/or deactivation.

The hydrogenation output, freed of catalyst where appropriate, will contain the desired beta amino ester together with the eliminated water, unreacted amine and small amounts of by-products. Small amounts are understood in this case to mean less than 5% by weight, preferably less than 3% by weight and more preferably less than 1% by weight of the compounds mentioned, based on the (catalyst-free) hydrogenation output.

This output may be worked up, using methods known in the art, to isolate and purify the beta amino ester. Mention in this regard may be made of extraction, evaporation, distillation and chromatography as suitable techniques. Upon isolation, it has been found that typical yields of the compound of Formula β-AE are at least 40% and often at least 60% or 70%.

The above described embodiments of the reductive amination process, whilst preferred, should not be construed as limiting of the present invention. A person of ordinary skill in the art may be aware of different catalysts and conditions under which reductive amination may occur. By way of example, alternative methods which might find utility in this invention are described inter alia in: M. Taibakhsh et. al. Synthesis, 2011, 490-496; and, S. Sato et al. Tetrahedron, 2004, 60, 7899-7906.

Coating, Adhesive, Sealant or Elastomer Compositions Derived from the Amino-Terminated Polymers or Oligomers

The amino-terminated polymers of the present invention can be used as reactive hardeners or curing agents for compositions based on compounds containing amine-reactive functionalities, including compositions based on mixtures of amine reactive functionalities. Such amine-reactive functionalities are well-known in the published literature and include: (i) activated unsaturated groups such as (meth)acryloyl groups and other groups derived from maleic acid and anhydride, fumaric acid, and itaconic acid and anhydride; (ii) activated methylene groups such as acetoacetate and malonate groups; (iii) epoxy groups; (iv) isocyanate groups; (v) aromatic activated aldehyde groups; (vi) cyclic carbonate groups; and, (vii) acid, anhydride, and ester groups, including oxalate esters. Broadly, such coating compositions should contain the amino terminated polymers in an amount such that there are from 0.25 to 4, for example from 0.5 to 2, equivalents of amino groups per equivalent of amine-reactive groups of the functionalized compounds.

It is at present envisaged that the amino-terminated polymers will find particular utility as hardeners or reactive curing agents for compositions comprising amine-reactive functionalities selected from epoxy groups, isocyanate groups and cyclic carbonate groups.

As examples of suitable epoxy groups-containing compounds may be mentioned: glycidyl ethers of (cyclo)aliphatic or aromatic hydroxyl compounds, such as ethylene glycol, butane glycol, glycerol, cyclohexane diol, mononuclear di- or polyvalent polyols, bisphenols such as Bisphenol-A or Bisphenol-F, and polynuclear phenols; epoxidized and optionally hydrogenated divinyl benzene; polyglycidyl ethers of phenol formaldehyde novolac; epoxy compounds containing an isocyanurate group; epoxidized polyalkadienes such as epoxidized polybutadiene; hydantoin epoxy resins; epoxy resins obtained by epoxidization of (cyclo)aliphatic alkenes such as dipentene dioxide, dicyclopentadiene dioxide and vinylcyclohexane dioxide; and, glycidyl group-containing resins such as polyesters, polyurethanes, polyepoxyesters and polyacrylics.

As examples of suitable isocyanate groups-containing compounds may be mentioned: (cyclo)aliphatic or aromatic polyisocyanates such as 1,2-propylene diisocyanate, trimethylene diisocyanate, tetramethylene diisocyanate, 2,3-butylene diisocyanate, hexamethylene diisocyanate, octamethylene diisocyanate, 2,2,4-trimethylhexamethylene diisocyanate, 2,4,4-trimethylhexamethylene diisocyanate, dodecamethylene diisocyanate, 1,3-cyclopentane diisocyanate, 1,2-cyclohexane diisocyanate, 1,4-cyclohexane diisocyanate, isophoron diisocyanate, 4-methyl-1,3-diisocyanatocyclohexane, trans-vinylidene diisocyanate, dicyclohexylmethane-4,4′-diisocyanate, 3,3′-dimethyldicyclohexylmethane-4,4′-diisocyanate, a toluene diisocyanate, 1,3-bis(isocyanatomethyl)benzene, a xylylene diisocyanate, 1,5-dimethyl-2,4-bis(isocyanatomethyl)benzene, 1,5-dimethyl-2,4-bis(2-isocyanatoethyl)benzene, 4,4′-diisocyanatodiphenyl, 3,3′-dichloro-4,4′-diisocyanatodiphenyl, 3,3′-diphenyl-4,4′-diisocyanatodiphenyl, 3,3′-dimethoxy-4,4′-diisocyanatodiphenyl methane, a diisocyanatonaphthalene; compounds such as 1,3,5-triisocyanatobenzene and 2,4,6-triisocyanatotoluene; the adduct of two molecules of a diisocyanate (such as hexamethylene or isophoron diisocyanate) with one molecule of a diol (such as ethylene glycol); the condensate of three molecules of a diisocyanate (such as hexamethylene diisocyanate) with one molecule of water; the adduct of three molecules of a diisocyanate (such as toluene or isophorone diisocyanate) with one molecule of trimethylolpropane; the adduct of 4 molecules of a diisocyanate (such as toluene diisocyanate) with one molecule of pentaerythritol; and, the isocyanurate trimer of a diisocyanate (such as hexamethylene diisocyanate).

As examples of suitable cyclic carbonate groups-containing compounds may be mentioned those produced by the addition of CO₂ to an epoxy groups-containing compound such as those mentioned above via any one of a number of well-known procedures. In this regards reference may be made to inter alia: U.S. Pat. No. 3,535,342; U.S. Pat. No. 4,835,289; U.S. Pat. No. 4,892,954; UK Patent No. GB 1485925; and EP-A-0119840.

The coating, adhesive or sealant compositions may, of course, also contain other standard additives such as pigments, fillers, levelling agents, foam suppressing agents, rheology control agents, catalysts, anti-oxidants, tackifiers, UV-stabilizers, and, minor amounts of co-solvents as required. The choice of appropriate additives is limited only in that these must be compatible with the other components of the coating composition.

### ILLUSTRATIVE EMBODIMENTS OF THE PRESENT INVENTION

An interesting but illustrative and non-limiting embodiment of the indirect amination synthesis of the present invention may be defined as a process for producing a β-amino ester functionalized oligomer or polymer, said process comprising the steps of:

a) providing a polyol which has a number average molecular weight (Mn) of from 300 to 10000 g/mol and which is represented by the formula A-(OH)_{q} wherein q is from 2 to 6 and A denotes an oligomeric or polymeric backbone with hetero atoms in the backbone or in pendent side chains, and converting said polyol into its corresponding acetoacetate functionalized compound by transacetoacetylation with an acetoacetate reagent represented by Formula 1,

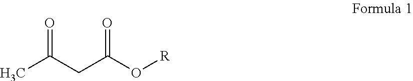

wherein R is a C1-C6 alkyl group;

b) converting said acetoacetate functionalized compound into its corresponding enamine by reaction with at least one amine represented by Formula 3,

R²R³NH Formula 3

wherein R² is hydrogen or a C1-C6 alkyl group; R³ is hydrogen or a C1-C18 aliphatic hydrocarbyl group which is optionally interrupted by one or more —N(R⁴)— groups of which R⁴ is a hydrogen atom; and, R² and R³ may form a ring together with the N-atom to which they are bound; and,

c) reducing the enamine product of step b) to form the corresponding β-amino ester functionalized compound.

An interesting but illustrative and non-limiting embodiment of the direct reductive amination synthesis of the present invention may be defined as a process for producing a β-amino ester functionalized oligomer or polymer, said process comprising the steps of:

a) providing a polyol which has a number average molecular weight (Mn) of from 300 to 10000 g/mol and which is represented by the formula A-(OH)_{q} wherein q is from 2 to 6 and A denotes an oligomeric or polymeric backbone with hetero atoms in the backbone or in pendent side chains, and converting said polyol into its corresponding acetoacetate functionalized compound by transacetoacetylation with an acetoacetate reagent represented by Formula 1,

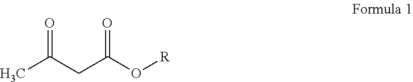

wherein R is a C1-C6 alkyl group;

d) converting said acetoacetate functionalized compound into its corresponding β-amino ester by a reductive amination with at least one amine represented by Formula 3,

R²R³NH Formula 3

wherein R² is hydrogen or a C1-C6 alkyl group; R³ is hydrogen or a C1-C18 aliphatic hydrocarbyl group which is optionally interrupted by one or more —N(R⁴)— groups of which R⁴ is a hydrogen atom; and, R² and R³ may form a ring together with the N-atom to which they are bound,

said process being characterized in that said reductive amination step is performed using an aluminium hydride or borohydride compound.

Various features and embodiments of the disclosure are described in the following examples, which are intended to be representative and not limiting.

### Examples

The following details are given for specific chemicals used in the Examples:

| | |
|---|---|
| Polyester 218: | Polyester polyol having a hydroxyl |
| | number of 133 mg KOH/g. |
| Tert-butyl acetoacetate | Purity ≥ 98 wt %; obtained from |
| | Lonza Group AG. |
| Baxxodur EC 252 | N-cyclohexyl-1,3-propanediamine, |
| | available from BASF. |

### Example 1

A flask with overhead stirring was charged with 254 g (602 mmol OH) of Polyester 218 and 100 g tert-butyl acetoacetate at room temperature under a nitrogen atmosphere. The flask was heated to 140° C. under a reflux condenser. After 4 hours of reaction, tert-butanol was removed under reduced pressure. Completion of the reaction was confirmed by the disappearance of the OH-band in an IR spectrum. The desired product (hereinafter AcAc1) was obtained as a colorless oil with a Brookfield viscosity of 1523 mPa·s at 25° C. (Spindle 27).

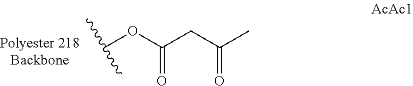 [SMILES: *OC(=O)CC(C)=O]

### Example 2

3.1 g (19.8 mmol) of Baxxodur EC 252 was added quickly to 10 g (19.8 mmol) of AcAc1 at room temperature under nitrogen and overhead stirring. After complete conversion—a period of 5 hours, as determined by thin layer chromatography—any remaining volatiles were removed in vacuo at 50° C. The desired product (En1) was obtained as a yellow oil, showing an amine content of 2.86% (amine value 107 mg KOH/g, determined by titration with 0.1N HCl). After three months of storage at room temperature, titration gave 2.722% amines (amine value mg KOH/g).

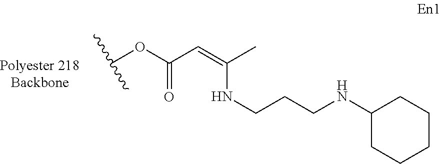 [SMILES: *OC(=O)/C=C(/C)NCCCNC1CCCCC1]

### Example 3

Step 1:

1.45 g (19.8 mmol) of n-butylamine was added quickly to 10.00 g (19.8 mmol) of AcAc1 at room temperature under nitrogen and overhead stirring. After complete conversion—approximately 5 hours, as determined by Thin Layer Chromatography (TLC) and amine titration—any remaining volatiles were removed in vacuo at 50° C. The enamine (En2) was obtained as a yellow oil showing an amine content of 0, as determined by titration with 0.1 N HCl.

Step 2:

2.79 g (4.97 mmol) of En2 was mixed with 0.30 g (4.97 mmol) of glacial acetic acid and stirred in an atmosphere in dry nitrogen. 1.58 g (7.45 mmol) of sodiumtriacetoxyborohydride (NaBH(OAc)₃) was added neat and the yellow slurry stirred at room temperature for 1 hour. 16 ml of tetrahydrofuran (THF) was added and the resulting suspension was stirred for a further 4 hours until complete consumption of En2 was determined by TLC. The reaction mixture was quenched by addition of 20 ml of a saturated solution of NaHCO₃ to give a pH of 8-9. 10 ml of diethyl ether (Et₂O) was added, the layers allowed to separate and the aqueous layer extracted (20 ml, Et₂O). The combined organic layers were washed with NaCl (10%, 10 ml), dried over MgSO₄ and the filtrate evaporated under reduced pressure. The desired product (βAE1) was obtained as a yellowish oil (2402 mg, c. 89% yield).

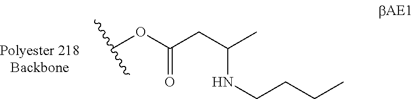 [SMILES: *OC(=O)CC(C)NCCCC]

### Example 4

1.45 g of n-butylamine (19.8 mmol) was added quickly to 10.02 g (19.8 mmol) of AcAc1 in THF (10 ml) at room temperature under nitrogen and overhead stirring. 6.28 g (26.8 mmol) of NaBH(OAc)₃ was added in one portion. After the foam formation ceased, 1.19 g (19.8 mmol) of glacial acetic acid was added and the colorless suspension stirred at room temperature for 16 hours. The reaction mixture was quenched by the addition of 50 ml of NaHCO₃ to give a pH of 8-9. 10 ml of diethyl ether (Et₂O) was added, the layers allowed to separate and the aqueous layer extracted (20 ml, Et₂O). The combined organic layers were washed with NaCl (10%, 10 ml). The organic phases were homogenized by adding THF (5 ml) and ethyl acetate (5 ml), dried over MgSO₄ and the filtrate evaporated under reduced pressure. The desired product (βAE1) was obtained as a yellowish oil (7581 mg, c. 76% yield).

In view of the foregoing description and examples, it will be apparent to those skilled in the art that equivalent modifications thereof can be made without departing from the scope of the claims.

### Storage Stability Tests

β-amino ester functionalized compounds (i.e. the products of an indirect or direct reductive amination) show an enhanced storage stability compared to their unreduced enamine precursors.

This can be exemplified with compound En1 from example 2 and its reduced derivative (β-amino ester):

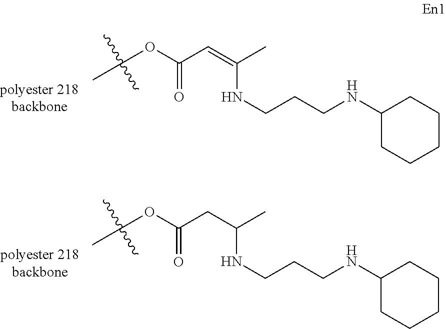 [SMILES: *OC(=O)/C=C(/C)NCCCNC1CCCCC1 ; *OC(=O)CC(C)NCCCNC1CCCCC1]

Immediately after synthesis, the amine values of both compounds were determined titrimetrically based on ASTM D2074: A defined amount of the polyester amine was dissolved in acetone and titrated with 0.1 N HCl versus bromothymol blue as indicator until a color change from blue (high pH) to yellow (acidic) was observed. The obtained amino values were defined as starting values.

Both compounds were stored in closed containers for 30 days at room temperature, after which the amine value were determined again. The β-amino ester showed a complete retention of the initial amine value (100%), whereas the amine value of En1 decreased to 96% of the initial amine value.

1. A process for producing a β-amino ester functionalized oligomer or polymer, said process comprising steps of:
providing a polyol represented by the formula A-(OH)_{q} wherein q≥2 and A denotes an oligomeric or polymeric backbone, and
converting said polyol into a corresponding acetoacetate functionalized compound by transacetoacetylation with an acetoacetate reagent; and
subjecting said acetoacetate functionalized compound to either indirect amination or direct reductive amination.

2. The process according to claim 1 for producing a β-amino ester functionalized oligomer or polymer, said process comprising steps of:
a) providing a polyol represented by the formula A-(OH)_{q} wherein q≥2 and A denotes an oligomeric or polymeric backbone, and converting said polyol into its corresponding acetoacetate functionalized compound by transacetoacetylation with an acetoacetate reagent;
b) converting said acetoacetate functionalized compound into its corresponding enamine by reaction with at least one amine bearing at least a primary or secondary amine group; and,
c) reducing the enamine product of step b) to form the corresponding β-amino ester functionalized compound.

3. The process according to claim 1 for producing a β-amino ester functionalized oligomer or polymer, said process comprising the steps of:
providing a polyol represented by the formula A-(OH)_{q} wherein q≥2 and A denotes an oligomeric or polymeric backbone A;
converting said polyol into its corresponding acetoacetate functionalized compound by transacetoacetylation with an acetoacetate reagent; and
converting said acetoacetate functionalized compound into its corresponding β-amino ester by a reductive amination with at least one amine bearing at least a primary or secondary amine group.

4. The process according to claim 1, wherein the acetoacetate reagent is represented by Formula 1: 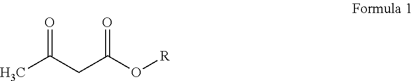
wherein R is a C1-C12 alkyl group.

5. The process according to claim 1, wherein A of said polyol denotes an oligomeric or polymeric backbone with hetero atoms in the backbone or in pendent side chains.

6. The process according to claim 1, wherein the hydroxyl functionality, q, of said polyol is from 2 to 6, and wherein said polyol has a number average molecular weight (Mn) of from 300 to 10000 g/mol.

7. The process according to claim 2, wherein said at least one amine is represented by Formula 3:
R²R³NH Formula 3
wherein: R² is hydrogen or a C1-C6 alkyl group;
R³ is hydrogen or a C1-C18 aliphatic hydrocarbyl group which is optionally interrupted by one or more —N(R⁴)— groups of which R⁴ is a hydrogen atom; and,
R² and R³ may form a ring together with the N-atom to which they are bound.

8. The process according to claim 7, wherein R² is hydrogen and R³ is a C1 to C12 alkyl group.

9. The process according to claim 7, wherein R² is hydrogen and, R³ is a C1 to C18 hydrocarbyl group, or a C1 to C12 hydrocarbyl group which is interrupted by one or more —N(R⁴)— groups of which R⁴ is a hydrogen atom.

10. The process according to claim 3, wherein said reductive amination step is performed using an aluminium hydride or borohydride compound.

11. The process according to claim 10, wherein said reductive amination is performed in the presence of:
1) a borohydride having the formula [(X)ₙBH₄₋n]⁻
wherein: n=0, 1, 2 or 3; and,
X is a cyano, acetoxy, trifluoroacetoxy, C1-C6 alkoxy or C1-C6 alkyl group;
or,
2) an aluminium hydride having the formula [(X)ₙAlH₄₋ₙ]⁻
wherein: n=0, 1, 2 or 3; and
X is a C1-C6 alkoxy or C1-C6 alkyl group.

12. The β-amino ester functional oligomer or polymer obtained according to the process of claim 1.

13. A β-amino ester functional oligomer or polymer having a primary amine level of less than 5 mg KOH/g; and a secondary amine level of from 5 to 599 mg KOH/g.

14. The β-amino ester functional oligomer or polymer according to claim 13, comprising a backbone polymer selected from the group consisting of: polyoxyalkylenes; polyesters; and, polycarbonates.

15. Use of the β-amino ester functional oligomer or polymer of claim 11 as a hardener or reactive curing agent for coating, adhesive, sealant or elastomer compositions based on compounds bearing amine-reactive functionalities, in particular compounds bearing amine-reactive functionalities selected from epoxy groups, isocyanate groups and cyclic carbonate groups.

16. The process according to claim 5, wherein A of said polyol denotes an oligomeric or polymeric backbone with hetero atoms in the backbone or in pendent side chains and selected from the group consisting of: polyoxyalkylene polyols; polyester polyols; polycarbonate polyols; and mixtures thereof.

17. The process according to claim 3, wherein said at least one amine is represented by Formula 3:
R²R³NH Formula 3
wherein: R² is hydrogen or a C1-C6 alkyl group;
R³ is hydrogen or a C1-C18 aliphatic hydrocarbyl group which is optionally interrupted by one or more —N(R⁴)— groups of which R⁴ is a hydrogen atom; and,
R² and R³ may form a ring together with the N-atom to which they are bound.

18. The process according to claim 17, wherein R² is hydrogen and R³ is a C1 to C12 alkyl group.

19. The process according to claim 17, wherein R² is hydrogen and, R³ is a C1 to C18 hydrocarbyl group, or a C1 to C12 hydrocarbyl group which is interrupted by one or more —N(R⁴)— groups of which R⁴ is a hydrogen atom.

20. A coating, adhesive, sealant or elastomer composition based on compounds bearing amine-reactive functionalities and containing a hardener or reactive curing agent comprising the β-amino ester functional oligomer or polymer of claim 11.

21. The coating, adhesive, sealant or elastomer composition of claim 20 wherein the amine-reactive functionalities of the compounds are selected from epoxy groups, isocyanate groups, cyclic carbonate groups and combinations thereof.
